(19) United States
(12) Patent Application Publication
(10) Pub. No.: US 2010/0284993 A1
(43) Pub. Date: Nov. 11, 2010

(54) Enhancing the Efficacy of Anti-Infective Therapeutics

(76) Inventor: Mitchell W. Mutz, La Jolla, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/739,390

(22) PCT Filed: Oct. 24, 2008
(86) PCT No.: PCT/US08/12130

§ 371 Date: Jul. 16, 2010

Related U.S. Application Data
(60) Provisional application No. 61/000,368, filed on Oct. 24, 2007.
(60) Provisional application No. 61/000,624, filed on Oct. 26, 2007.

(51) Int. Cl. A61K 38/46; A61K 38/00; A61K 31/397; A61K 31/4025; A61K 31/501; A61K 31/41; A61K 31/407; A61K 31/335

(52) U.S. Cl. 424/94.6; 514/1.1; 514/210.1; 514/210.13; 514/256; 514/359; 514/411; 514/450

(57) ABSTRACT

In an embodiment of the invention, a method of enhancing the efficacy of an anti-infective therapeutic agent against an obligate or facultative intracellular parasite in a host is provided. The method comprises administering to the host an effective amount of a bifunctional compound of less than about 5000 Daltons comprising the therapeutic or an active derivative, fragment or analog thereof and a protein binding moiety. The protein binding moiety binds to at least one intracellular protein. The bifunctional compound has a higher intracellular concentration as compared to the therapeutic itself in order to enhance the efficacy of the anti-infective therapeutic against the obligate or facultative intracellular parasite.

### CROSS-REFERENCE TO RELATED APPLICATIONS

This application claims priority to U.S. Provisional Applications Nos. 61/000,368, filed Oct. 24, 2007, and 61/000,624, filed Oct. 26, 2007. These applications are incorporated by reference herein in their entirety.

### TECHNICAL FIELD

This invention relates generally to pharmacology and more specifically to the modification of known active agents to give them more desirable properties.

### BACKGROUND

Many anti-infective therapeutics including both antifungals and antibacterials have limited efficacy against obligate or facultative intracellular parasites. In infections of Salmonella, Listeria monocytogenes, Staphylococcus aureus, Candida albicans, Aspergillus fumigatus, and Cryptococcus neoformens, intracellular infection can make control and eradication of the infection problematic (Tulkens, P. M., Eur. J. Clin. Microbial Infect. Dis., 10, 1991, pp 100-106). Antibiotics such as beta-lactams do not have significant intracellular accumulation in phagocytic cells which are a common site of intracellular infection. In addition, many antifungals may not have much mammalian intracellular accumulation or are not retained well intracellularly, and thereby have compromised efficacy against intracellular parasites (S. Ballesta et al., J. Antimicrob. Chemother. 55, 2005, pp 785-787). However, macrolide-based antibiotics can enhance intracellular accumulation of anti-infectives and improve efficacy against intracellular parasites (Tulkens, supra).

A second problem with many antibiotics is short (less than three hour) elimination half life which limits therapeutic efficacy. For example, meropenem, a broad spectrum antibiotic, has an elimination half life of one hour. It has been widely demonstrated that maintaining levels of this and other antibiotics above the minimum inhibitory concentration in a sustained manner by continuous infusion provides superior efficacy vs. dosing three or four times per day (E. Viaene et al., Antimicrob. Agents Chemother., 46, 2002, pp 2327-2332). However, continuous infusion is problematic since it can involve installing an additional i.v. line to avoid physiochemical interactions with other administered drugs. Furthermore, meropenem and other anti-infectives are unstable at 20-37° C., requiring a new infusion bag every hour. This also makes a continuous infusion strategy technically difficult. An improved meropenem with a longer elimination half-life and better intracellular distribution would create a drug with substantially improved properties (Wolfgang Krueger et al., Antimicrob. Agents Chemother., 49, 2005, pp 1881-1889).

Attempts to make a longer lasting version of meropenem via conventional medicinal chemistry techniques have not been able to preserve the broad-spectrum anti-infective activity of meropenem. For example, ertapenem is a derivative of meropenem with a longer half life of four hours. However, ertapenem achieves this longer half life via albumin binding and, moreover, is not effective against Pseudomonas aeruginosa, unlike meropenem. In addition, in the albumin-bound state, ertapenem is not available to bind to the drug target. Lastly, attempts to improve the half-life and area under the curve of anti-infectives using nano-encapsulation have only been marginally successful due to the failure to target different strains of infectious agents such as Pseudomonas aeruginosa (Z. Drulis-Kawa et al., Int. J. Pharmaceutics, 315, 2006, pp. 59-66).

The triazoles are a class of antifungals with broad spectrum activity and are a relatively new drug class. Several members of this class, such as voriconazole, are extensively metabolized by cytochrome P450 enzymes CYP2C19, CYP2C9 and CYP3A4. Unfortunately, this extensive metabolism makes administering triazoles with other drugs that are inhibitors or inducers of P450 enzymes problematic. Lowering the P450 metabolism to allow safer co-administration with other therapeutics would be a desirable improvement for this drug class, especially if combined with increased intracellular exposure to gain efficacy against intracellular parasites. Currently, the effective dose of voriconazole can vary by a factor of 10 or more depending on which drugs are co-administered with voriconazole and the patient's ability to metabolize voriconazole via cytochrome P450 (Y. Ikeda, Clin Pharm. & Ther., 75, 2004, pp 586-588). Co-administration of the triazoles with other therapeutic agents is often essential to maintain patient health since invasive fungal infections often occur in patients in an immunocompromised state due to severe illnesses such as HIV infection and cancer.

Efficacy of pharmaceutical agents can be improved by the initial loading into blood cells. Such loading can serve to lower the maximum plasma concentration (Cₘₐₓ) and increase the area under the curve for the drug to create a sustained release effect. For example, clinicians have used direct erythrocyte loading to enhance compound half-life, as well as reduce toxicity, and as a means to target a therapeutic more effectively (M. Magnani et al., Gene Therapy 9, 749-751 (2002)). The method disclosed by Magnani et al. requires the patient to donate blood for therapeutic loading, and then wait several hours until the re-introduction of the treated blood cells. Benefits of this approach were improved clinical response to the steroid, dexamethasone, and lower toxicity. The mechanism for the improvement appeared to be a substantial increase in the area under the curve and a substantially prolonged release relative to dexamethasone administered via injection or by other means. However, the method of extracting and then re-introducing blood to the patient carries risks of infection, is also painful, and requires a treatment of approximately three hours per dose.

There continues to be a need for anti-infectives which are more effective against facultative and obligate intracellular parasites.

### SUMMARY OF THE INVENTION

In an embodiment of the invention, a method of enhancing the efficacy of an anti-infective therapeutic agent against an obligate or facultative intracellular parasite in a host is provided. The method comprises administering to the host an effective amount of a bifunctional compound of less than about 5000 Daltons comprising the therapeutic or an active derivative, fragment or analog thereof and a protein binding moiety. The protein binding moiety binds to at least one intracellular protein. The bifunctional compound has a higher intracellular concentration as compared to the therapeutic itself in order to enhance the efficacy of the anti-infective therapeutic against the obligate or facultative intracellular parasite.

In a further embodiment of the invention, a bifunctional compound comprises a drug moiety, a linker moiety, and a recruiter moiety. The linker is chosen to enhance the solubility of the drug moiety relative to its free drug form. The recruiter is a biomoiety and may consist of protein, carbohydrate, RNA, DNA, or lipid.

In a further embodiment of the invention, a bifunctional compound comprises a drug moiety, a linker moiety, and a recruiter moiety. The bifunctional compound is administered together with a free form of either the drug moiety or the recruiter moiety.

### FIGURES

FIG. 1 depicts the structure of SLF linked to a modular linker and target binding moiety, for example a paclitaxel therapeutic. Due to the modular nature of the synthesis, the linker group and target-binding group may be readily altered.

FIG. 2 illustrates how the steric bulk of a protein can confer protection from enzymes.

FIG. 3 shows results of an experiment where intracellular sequestration protects a bifunctional compound from degradation by an extracellular esterase. Briefly, a bifunctional protease inhibitor containing an ester moiety is incubated in either whole blood or plasma only and the amount of remaining compound vs time is assessed by periodically collecting samples, performing an organic extraction, and quantitating the remaining compound by LC-MS. In addition, the % of compound remaining intracellularly and extracellularly is also assessed by LC-MS in the whole blood sample. The rate of degradation of bifunctional compound is actually slowed by the addition of whole blood with cells compared to the rate of degradation in pure plasma without cells. The cells provide a site of intracellular sequestration and protection from an esterase present primarily in plasma.

In FIG. 4, the left side depicts the bimodal binding character of FK506 whereby it binds both FKBP and calcineurin. The schematic on the right depicts how the calcineurin-binding mode can be eliminated by substituting a linker and target binding moiety. In this manner, FK506 can simultaneously target FKBP and bind a second protein. Synthetic ligands with no affinity for calcineurin such as SLF may also be used.

In FIG. 5, (A) shows the structure of FK506 bound to curcumin. (B) illustrates that FK506-curcumin is protected from CYP3a4, a P450 enzyme, in the presence of FKBP. (C) gives a schematic of the Invitrogen assay used.

In FIG. 6A, the left side illustrates sample linkers that could be employed in a modular synthetic scheme.

FIG. 6B is exemplary for a type of polar linker which can be used to increase bifunctional solubility in aqueous solutions relative to the parent drug compound or recruiter moiety.

FIG. 7 exhibits a synthetic scheme for a bifunctional form of ertapenem, an anti-bacterial therapeutic.

FIG. 8 illustrates the efficacy of a bifunctional paclitaxel drug in cell culture without drug-degrading enzymes. Lower o.d. 540 indicates more tumor cell growth inhibition by paclitaxel-SLF (right bar in each pair).

FIG. 9 shows the difference in partitioning between the extra- and intracellular space due to the presence of the recruiter moiety in an in vivo mouse model study. It may be seen that extra vs intracellular distribution is altered by addition of a ligand for a non-target protein.

FIG. 10 shows the effect of area under the curve for a bifunctional compound in mice vs. a monofunctional compound. Compound was administered via a tail vein injection to mimic intravenous drug administration. Data shows 25 fold increase in area under the curve for the bifunctional vs. the monofunctional.

FIG. 11 shows the efficacy of the paclitaxel bifunctional in a xenograft tumor mouse model vs a vehicle control containing the Cremaphor-ethanol solvent only. The figure depicts the ability of paclitaxel bifunctional to slow growth of MDA-MB-435 Xenograft tumor cells relative to control.

FIG. 12 shows the partitioning of a protease inhibitor bifunctional between blood cells and plasma. Extra vs intracellular distribution is altered by addition of ligand for non-target protein. Final drug concentration measured after 5 hours in mouse blood at 37° C.

FIG. 13 shows that the addition of cells allows sequestration from P450 enzymes and protects a curcumin-SLF bifunctional from intracellular degradation. The rate of hepatic metabolism is reduced for bifunctional curcumin vs. curcumin in an in vitro assay of cytp450 metabolism where higher fluorescence indicates reduced cytp450 activity. The presence of liver microsomes provides both a source of cytp450 and a opportunity for intracellular sequestration of compound.

### DETAILED DESCRIPTION OF PREFERRED EMBODIMENTS

Before describing the present invention in detail, it is to be understood that this invention is not limited to specific solvents, materials, or device structures, as such may vary. It is also to be understood that the terminology used herein is for the purpose of describing particular embodiments only, and is not intended to be limiting.

As used in this specification and the appended claims, the singular forms “a,” “an,” and “the” include both singular and plural referents unless the context clearly dictates otherwise. Thus, for example, reference to “an active ingredient” includes a plurality of active ingredients as well as a single active ingredient, reference to “a temperature” includes a plurality of temperatures as well as single temperature, and the like.

The term “intracellular protein” encompasses proteins which are found primarily in the intracellular space and also transmembrane proteins or receptors.

Where FK506 is used, variants or analogs of FK506 are included, such as rapamycin, pimecrolimus, or synthetic ligands of FK506 binding proteins (SLFs) such as those disclosed in U.S. Pat. Nos. 5,665,774, 5,622,970, 5,516,797, 5,614,547, and 5,403,833 or described by Holt et al., “Structure-Activity Studies of Synthetic FKBP Ligands as Peptidyl-Prolyl Isomerase Inhibitors,” Bioorganic and Medicinal Chemistry Letters, 4 315-320 (1994) or other moieties which bind to peptidyl prolyl isomerases.

In an embodiment of the invention, the efficacy of an anti-microbial, particularly one directed against an obligate or facultative intracellular parasite, may be improved by coupling it to a ligand which can bind to an intracellular protein. The coupling results in a bifunctional molecule.

The bifunctional molecules of the invention are generally described by the formula:

X-L-Z

wherein:

X is a drug moiety;

L is a bond or linking group; and

Z is a recruiter moiety.

The bifunctional compounds are typically small. As such, the molecular weight of the bifunctional compound is generally at least about 100 D, usually at least about 400 D and more usually at least about 500 D. The molecular weight may be less than about 800 D, about 1000 D, about 1200 D, or about 1500 D, and may be as great as 2000 D or greater, but usually does not exceed about 5000 D. The preference for small molecules is based in part on the desire to facilitate oral administration of the bifunctional compound. Molecules that are orally administrable tend to be small.

Bifunctional compound in general have aroused considerable interest in recent years. See, for example, U.S. Pat. Nos. 6,270,957, 6,316,405, 6,372,712, 6,887,842, and 6,921,531 and Patent Cooperation Treaty publication WO2007/053792.

The moiety X in the bifunctional compound will generally be derived from a known anti-infective drug. The moiety Z will generally be chosen to be a moiety which can bind to an intracellular protein.

In general, the moiety X can be derived from a wide category of anti-infective drugs which have some effect against intracellular parasites. Such parasites include, for example, viruses in general (which commonly need to be inside a cell to reproduce), Chlamydia spp., Rickettsia spp., Listeria monocytogenes, Mycobacterium spp., Salmonella typhimurium, Yersinia pestis, Listeria spp., Legionella pneumophila, Cryptococcus neoformans, Candida albicans, Aspergillus fumigatus, Histoplasma spp., Leishmania spp., and Trypanosoma spp. The therapeutic target may be any kind of parasitic organism (viral, bacterial, yeast, fungal, amoebal, plasmodial, etc.) which occupies a host organism to survive and has a harmful effect on the host organism.

It is possible that a moiety X could be derived from an anti-infective not currently used against intracellular parasites. The increased intracellular concentration achievable by means of the invention might allow a bifunctional molecule containing such an anti-infective to be used against such parasites.

Thus for example, moiety X may be derived from 2,4-diaminopyrimidines, acedapsone, acetosulfone sodium, acetyl sulfamethoxypyrazine, acyclovir, alamecin, alexidine, amdinocillin, amdinocillin pivoxil, amicycline, amifloxacin, amifloxacin mesylate, amikacin, amikacin sulfate, aminosalicylate sodium, aminosalicylic acid, amoxicillin, amphomycin, ampicillin, ampicillin sodium, apalcillin sodium, apicycline, apramycin, arbekacin, aspartocin, astromicin sulfate, avilamycin, avoparcin, azidamfenicol, azithromycin, azlocillin, azlocillin sodium, aztreonam, bacampicillin, bacampicillin hydrochloride, bacitracin, bacitracin methylene disalicylate, bacitracin zinc, bambermycins, benzoylpas calcium, benzylpenicillin sodium, benzylpenicillinic acid, benzylsulfamide, berythromycin, betamicin sulfate, biapenem, biniramycin, biphenamine hydrochloride, bispyrithione magsulfex, brodimoprim, butikacin, butirosin, butirosin sulfate, capreomycin, capreomycin sulfate, carbadox, carbenicillin disodium, carbenicillin indanyl sodium, carbenicillin phenyl sodium, carbenicillin potassium, carbomycin, carumonam, carumonam sodium, cefaclor, cefadroxil, cefamandole, cefamandole nafate, cefamandole sodium, cefaparole, cefatrizine, cefazaflur sodium, cefazedone, cefazolin, cefazolin sodium, cefbuperazone, cefdinir, cefepime, cefepime hydrochloride, cefetecol, cefixime, cefmetazole, cefmetazole sodium, cefminox, cefmnenoxime hydrochloride, cefonicid monosodium, cefonicid sodium, cefoperazone sodium, ceforanide, cefotaxime sodium, cefotetan, cefotetan disodium, cefotiam hydrochloride, cefoxitin, cefoxitin sodium, cefozopran, cefpimizole, cefpimizole sodium, cefpiramide, cefpiramide sodium, cefpirome, cefpirome sulfate, cefpodoxime proxetil, cefprozil, cefroxadine, cefsulodin sodium, ceftazidime, ceftibuten, ceftizoxime sodium, ceftriaxone sodium, cefuroxime, cefuroxime axetil, cefuroxime pivoxetil, cefuroxime sodium, cephacetrile sodium, cephalexin, cephalexin hydrochloride, cephaloglycin, cephaloridine, cephalothin sodium, cephapirin sodium, cephradine, cetocycline hydrochloride, cetophenicol, chloramphenicol, chloramphenicol palmitate, chloramphenicol pantothenate complex, chloramphenicol sodium succinate, chlorhexidine phosphanilate, chloroxylenol, chlortetracycline, chlortetracycline bisulfate, chlortetracycline hydrochloride, cinoxacin, ciprofloxacin, ciprofloxacin hydrochloride, cirolemycin, clarithromycin, clinafloxacin, clinafloxacin hydrochloride, clindamycin, clindamycin hydrochloride, clindamycin palmitate hydrochloride, clindamycin phosphate, clofazimine, clomocycline, cloxacillin benzathine, cloxacillin sodium, cloxyquin, colistimethate sodium, colistin, colistin sulfate, coumermycin, coumermycin sodium, cyclacillin, cycloserine, dalfopristin, dapsone, daptomycin, demeclocycline, demeclocycline, demeclocycline hydrochloride, demecycline, denofungin, diathymosulfone, diaveridine, dibekacin, dicloxacillin, dicloxacillin sodium, dihydrostreptomycin sulfate, dipyrithione, dirithromycin, doripenem, doxycycline, doxycycline calcium, doxycycline fosfatex, doxycycline hyclate, droxacin sodium, echinocandins such as caspofungin, micafungin, and anidulafungin, enduracidin, enoxacin, enviomycin, epicillin, epitetracycline hydrochloride, ertapenem, erythromycin, erythromycin acistrate, erythromycin estolate, erythromycin ethylsuccinate, erythromycin gluceptate, erythromycin lactobionate, erythromycin propionate, erythromycin stearate, ethambutol hydrochloride, ethionamide, famciclovir, fenbenicillin, fleroxacin, flomoxef, florfenicol, floxacillin, fluconazole, fludalanine, flumequine, fosfomycin, fosfomycin tromethamine, fumoxicillin, furaltadone, furazolium chloride, furazolium tartrate, fusidate sodium, fusidic acid, ganciclovir, gatifloxacin, gentamicin sulfate, gloximonam, glucosulfone sodium, gramicidin, grepagloxacin, haloprogin, hetacillin, hetacillin potassium, hexedine, ibafloxacin, imipenem, isepamicin, isoconazole, isoniazid, itraconazole, josamycin, kanamycin sulfate, ketoconazole, kitasamycin, levofloxacin, levofuraltadone, levopropylcillin potassium, lexithromycin, lincomycin, lincomycin hydrochloride, lincosamides, linezolid, lomefloxacin, lomefloxacin hydrochloride, lomefloxacin mesylate, loracarbef, macrolides, mafenide, meclocycline, meclocycline sulfosalicylate, megalomicin potassium phosphate, mequidox, meropenem, methacycline, methacycline hydrochloride, methenamine, methenamine hippurate, methenamine mandelate, methicillin sodium, metioprim, metronidazole hydrochloride, metronidazole phosphate, mezlocillin, mezlocillin sodium, minocycline, minocycline hydrochloride, mirincamycin hydrochloride, monensin, monensin sodium, moxalactam, mupirocin and tuberin, nafcillin sodium, nalidixate sodium, nalidixic acid, natamycin, nebramycin, neomycin, neomycin palmitate, neomycin sulfate, neomycin undecylenate, netilmicin, netilmicin sulfate, neutramycin, nifuradene, nifuraldezone, nifuratel, nifuratrone, nifurdazil, nifurimide, nifurpirinol, nifurquinazol, nifurthiazole, nitrocycline, nitrofurans, nitrofurantoin, nitromide, noprylsulfamide, norfloxacin, novobiocin sodium, ofloxacin, ormetoprim, oxacillin sodium, oximonam, oximonam sodium, oxolinic acid, oxytetracycline, oxytetracycline calcium, oxytetracycline hydrochloride, paldimycin, parachlorophenol, paromomycin, paulomycin, pefloxacin, pefloxacin mesylate, penamecillin, penethamate hydriodide, penicillin 0, penicillin G benzathine, penicillin G potassium, penicillin G procaine, penicillin G sodium, penicillin o-benethamine, penicillin V, penicillin V benzathine, penicillin V hydrabamine, penicillin VK, penicillin V potassium, penimepicycline, pentizidone sodium, phenethicillin potassium, phenyl aminosalicylate, phthalylsulfacetamide, piperacillin sodium, pirbenicillin sodium, piridicillin sodium, pirlimycin hydrochloride, pivampicillin hydrochloride, pivampicillin pamoate, pivampicillin probenate, polymyxin b sulfate, porfiromycin, propikacin, pyrazinamide, pyrithione zinc, quindecamine acetate, quinolones and analogs thereof, quinupristin, racephenicol, ramoplanin, ranimycin, relomycin, repromicin, ribostamycin, rifabutin, rifametane, rifamexil, rifamide, rifampin, rifapentine, rifaximin, rolitetracycline, rolitetracycline nitrate, rosaramicin, rosaramicin butyrate, rosaramicin propionate, rosaramicin sodium phosphate, rosaramicin stearate, rosoxacin, roxarsone, roxithromycin, sancycline, sanfetrinem sodium, sarmoxicillin, sarpicillin, scopafingin, sisomicin, sisomicin sulfate, solasulfone, sparfloxacin, spectinomycin, spectinomycin hydrochloride, spiramycin, stallimycin hydrochloride, steffimycin, streptomycin sulfate, streptonicozid, sulfabenz, sulfabenzamide, sulfacetamide, sulfacetamide sodium, sulfachrysoidine, sulfacytine, sulfadiazine, sulfadiazine sodium, sulfadoxine, sulfalene, sulfamerazine, sulfameter, sulfamethazine, sulfamethizole, sulfamethoxazole, sulfamonomethoxine, sulfamoxole, sulfanilate zinc, sulfanitran, sulfasalazine, sulfasomizole, sulfathiazole, sulfazamet, sulfisoxazole, sulfisoxazole acetyl, sulfisoxazole diolamine, sulfomyxin, sulfonamides, sulfones, sulopenem, sultamicillin, suncillin sodium, talampicillin hydrochloride, teicoplanin, temafloxacin hydrochloride, temocillin, tetracycline, tetracycline hydrochloride, tetracycline phosphate complex, tetroxoprim, thiamphenicol, thiphencillin potassium, ticarcillin cresyl sodium, ticarcillin disodium, ticarcillin monosodium, ticlatone, tigemonam, tiodonium chloride, tobramycin, tobramycin sulfate, tosufloxacin, trimethoprim, trimethoprim sulfate, trisulfapyrimidines, troleandomycin, trospectomycin sulfate, tyrothricin, undecylenate, valacyclovir, vancomycin, vancomycin hydrochloride, virginiamycin, voriconazole, or zorbamycin.

Reference is made to Laurence L. Brunton et al., Goodman & Gilman's The Pharmacological Basis of Therapeutics (11th ed. 2005) for information about anti-infectives and other drugs which may be candidates from which the drug moiety X can derive.

The drug moiety X will preferably derive from an antibiotic which is known to have a low intracellular accumulation. The intracellular accumulation of a variety of antibiotics has been characterized. For example, it is known that β-lactams, triazoles, cephalosporinase, aminoglycosides, ansamycins, and tetracyclines have particularly low intracellular accumulation or intracellular efficacy, and that within other broad classes such as glycopeptides, fluoroquinolones, and macrolides there are at least some members with quite low intracellular accumulation. Linezolid has also been found to have an unusually low intracellular accumulation.

Intracellular accumulation is commonly expressed as a ratio between intracellular and extracellular concentration. It may depend on the particular cell in which the evaluation is taking place. THP-1 macrophages in cell culture may, for example, be used to evaluate intracellular accumulation. Drug moieties for which the bifunctional strategy of this invention is particularly appropriate may include, for example, those with an intracellular accumulation ratio of less than about 10, less than about 5, less than about 4, less than about 3, less than about 2, or less than about 1.

The moiety X may be obtained modifying an existing or known anti-infective drug by a variety of chemical techniques. It will often be preferable for the modification to be minimal, e.g., for the linker or bond connecting the moiety X to Z to substitute for a hydrogen within the free anti-infective drug.

The synthesis of the bifunctional compound starts with a choice of suitable drug and recruiter moieties. It is desirable to identify on each of these moieties a suitable attachment point which will not result in a loss of biological function for either one. This may be done based on the existing knowledge of what modifications result or do not result in a biological function. On that basis, it may reliably be conjectured that certain attachment points on the pharmacokinetic and drug moieties do not affect biological function. Likewise, in FIG. 7 one sees a secondary amine functions on SLF, which can serve as an attachment point to the anti-infective ertapenem.

A general synthetic strategy is to locate a secondary amine on the drug moiety at which the drug moiety can be split (so that the secondary amine does not form part of any cycle in the drug moiety). The secondary amine is chosen such that, from experimental or other considerations, it is believed that the drug will retain its efficacy if only the portion of the drug moiety to one side of the secondary amine is present. The portion of the drug moiety to that side of the secondary amine is then synthesized by any appropriate technique, with the secondary amine in the synthesized molecule being protected during synthesis by an appropriate protecting moiety such as Boc. The protecting moiety is then removed, leaving a primary amine which may react with a carboxyl group through a variety of known chemistries for making a peptide bond (see, e.g., J. Mann et al., Natural Products: Their Chemistry and Biological Significance (1994), chapter 3).

In general, the recruiter moiety Z will have a molecular weight less than about 2000 D, less than about 1800 D, less than about 1500 D, less than about 1100 D, or less than about 900 D.

The recruiter biomoiety Z may bind to protein within a host or parasite cell. In certain embodiments, lipid, nucleic acid, carbohydrate, or any component of such a cell. The presence of the recruiter preferably causes a rapid distribution of the bifunctional drug inside a mammalian cell. This is helped if the recruiter moiety Z is lipophilic. The rapid distribution into the intracellular space has several effects: the anti-infective is in proximity to intracellular parasites, the anti-infective is protected from extracellular metabolism, and area under the curve is increased since release is slowed from inside cells due to the affinity of the recruiter moiety for the intracellular recruiter target. The rapid distribution into the intracellular space also lowers the maximum plasma concentration of the drug, and gives correspondingly less exposure to the extracellular cytochrome P450 enzymes and other extracellular enzymes which can metabolize drugs such as peptidases, proteases, hydrolases, aldolases, and esterases.

The increased presence inside the cell of the bifunctional relative to the parent compound (e.g., the drug from which the drug moiety derives) increases the efficacy of the drug against obligate and facultative intracellular parasites. Moreover, if the bifunctional contains a recruiter with increased affinity for a biomoiety unique or highly abundant in the parasite, the bifunctional will be preferentially targeted to the parasite. Specific recruiters with higher affinity for parasite proteins vs. mammalian proteins have been described. For example, L-685,818 is a peptidyl prolyl isomerase inhibitor which preferentially binds to peptidyl prolyl isomerases of the protozoan parasite Trypanosoma cruzi over human FKBP (A. Moro et al., EMBO Journal, 14 (11), pp 2483-2490, 1995). This selection is possible due to sequence divergence of peptidyl prolyl isomerase among various organisms (A. Galat, Eur. J. Biochem. 267, pp 4945-4959, 2000). Moro (supra) also describes that a peptidyl prolyl isomerase binder reduces the ability of pathogens to infect mammalian cells. In addition, if a short linker between drug and recruiter moieties are used, the drug toxicity may be reduced in the host mammalian cell vs. the parasite cell (P. Braun et al., J. Am. Chem. Soc., 125, pp. 7575-7580, 2003).

The recruiter moiety Z may be any moiety which binds to intracellular proteins. For example, Z may bind to a peptidyl prolyl isomerase. Alternatively, the recruiter moiety may bind heat shock proteins or other chaperone proteins, whose function is intracellular. Heat shock proteins have the possible advantage of higher expression in inflammation.

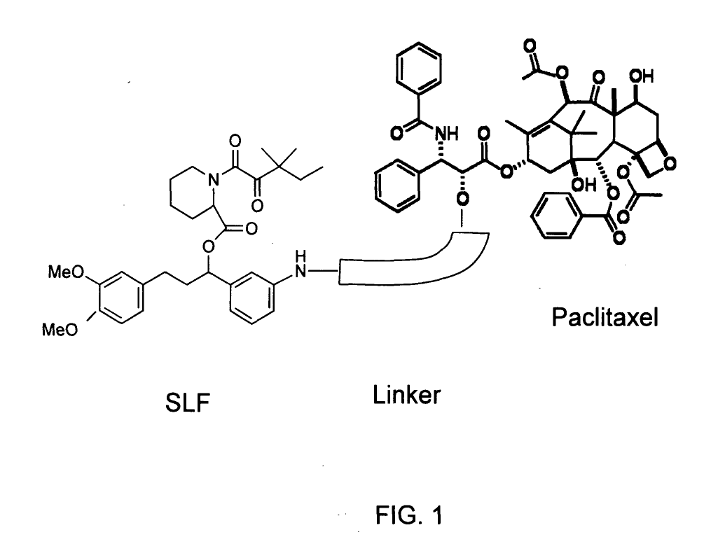

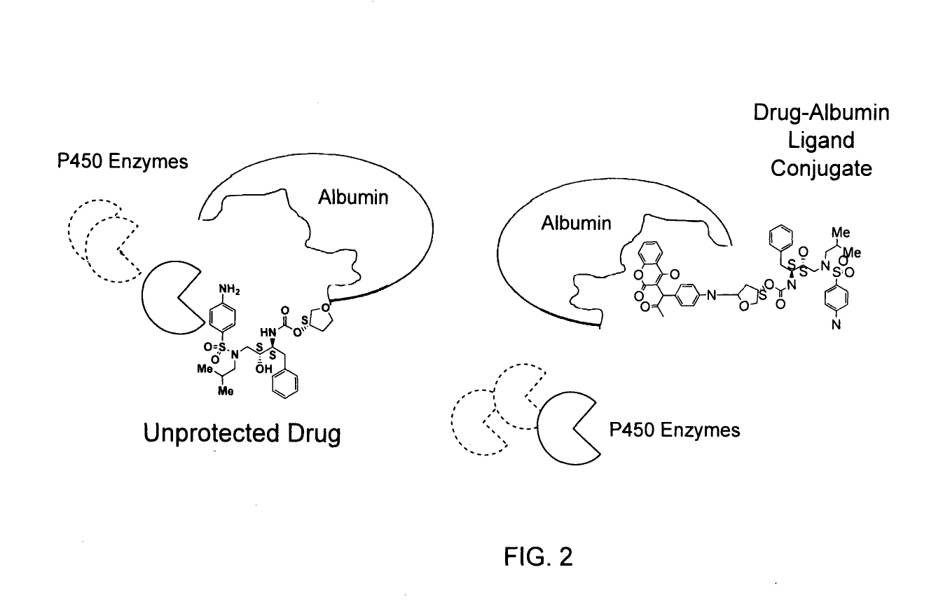

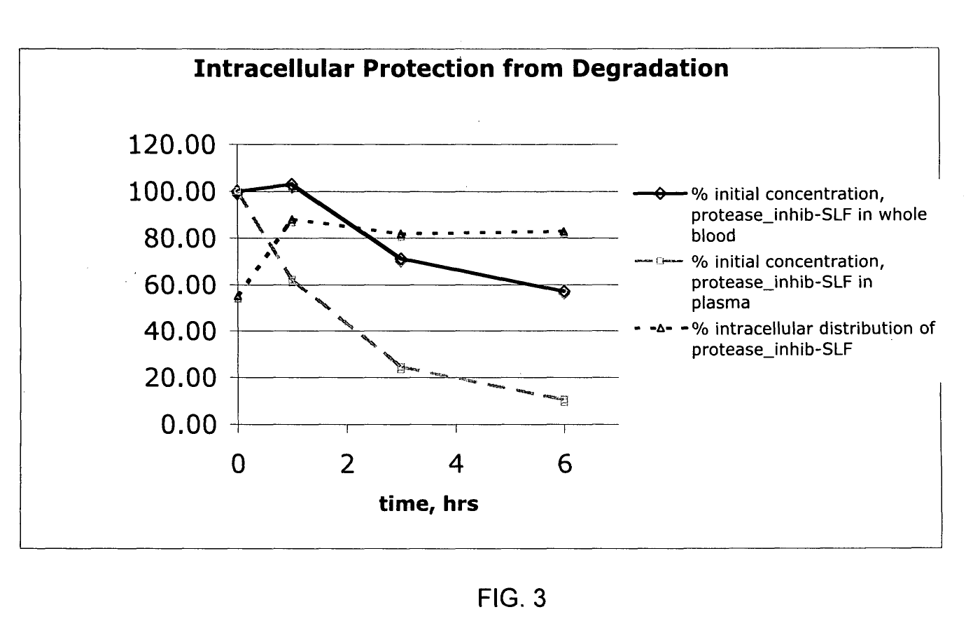

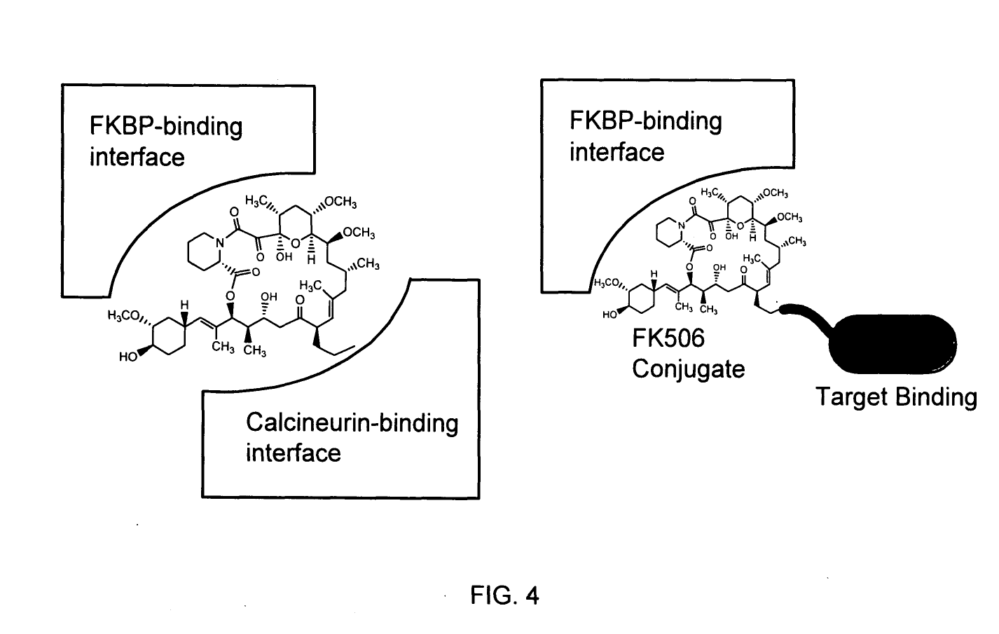

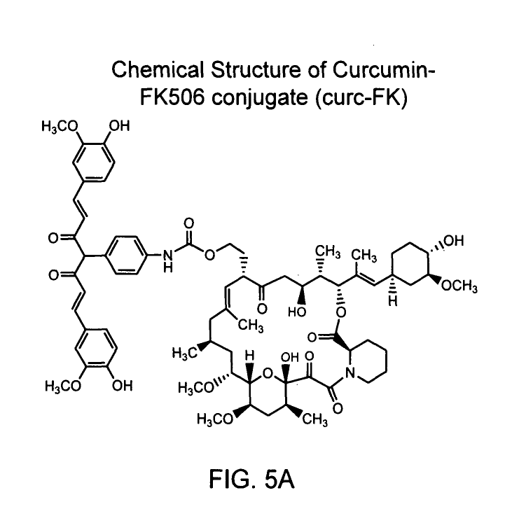

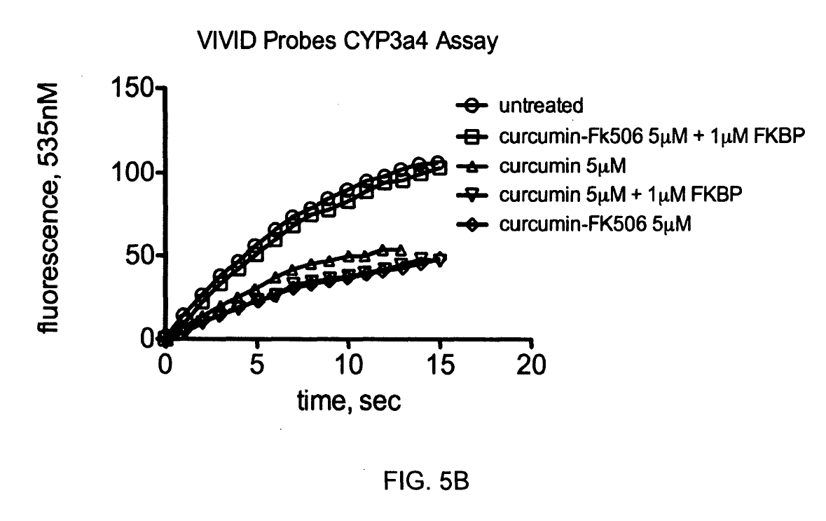

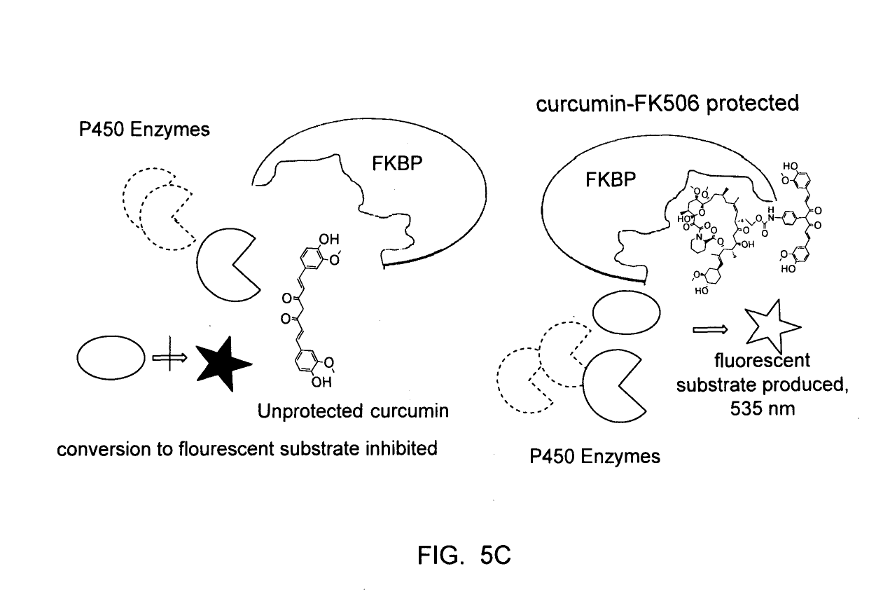

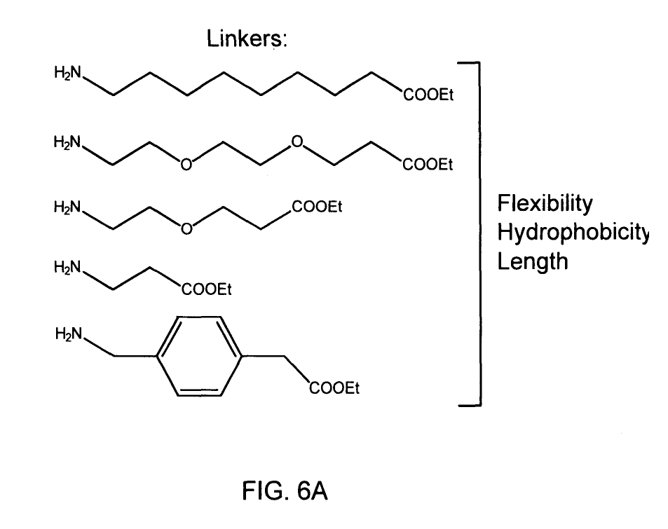

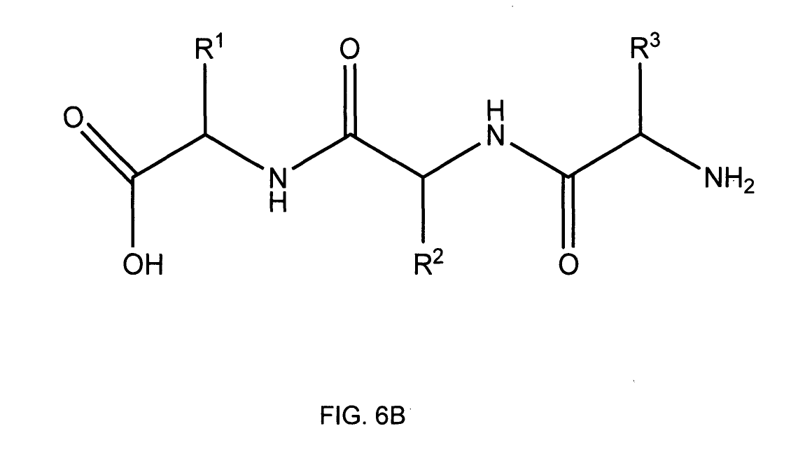

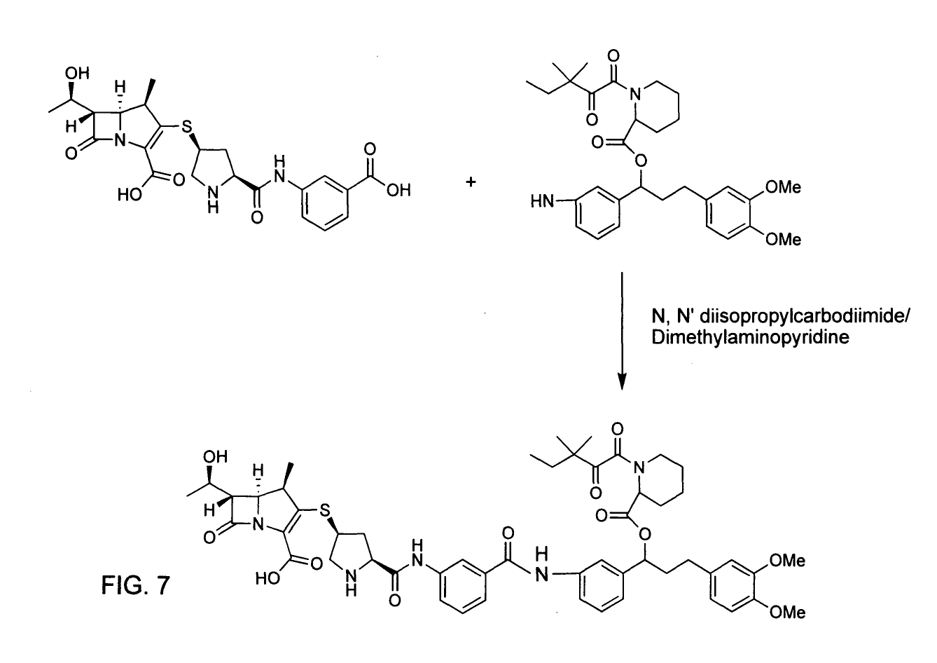

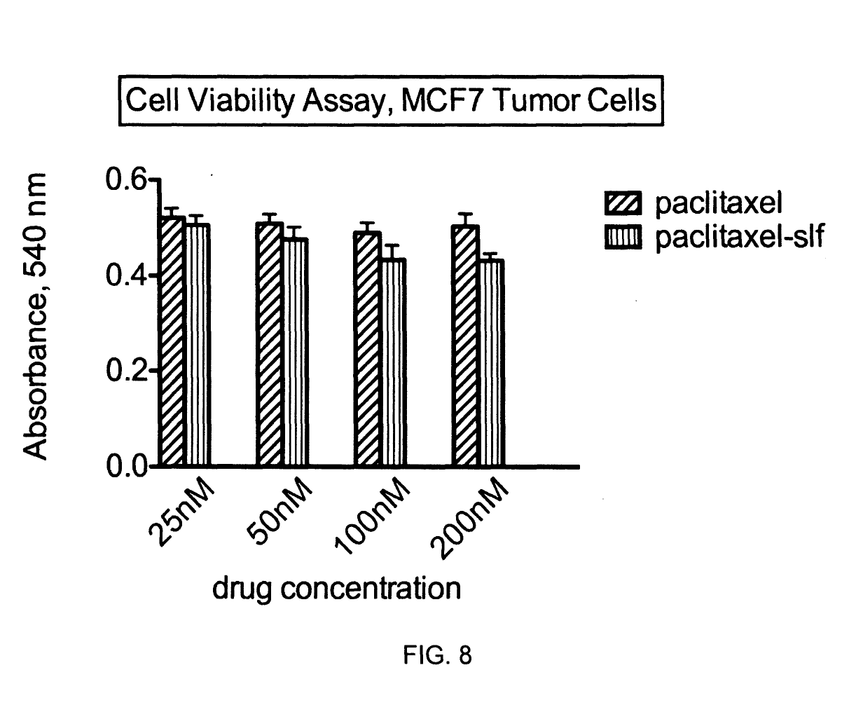

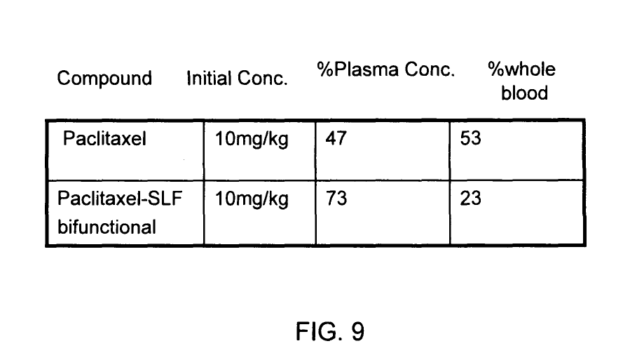

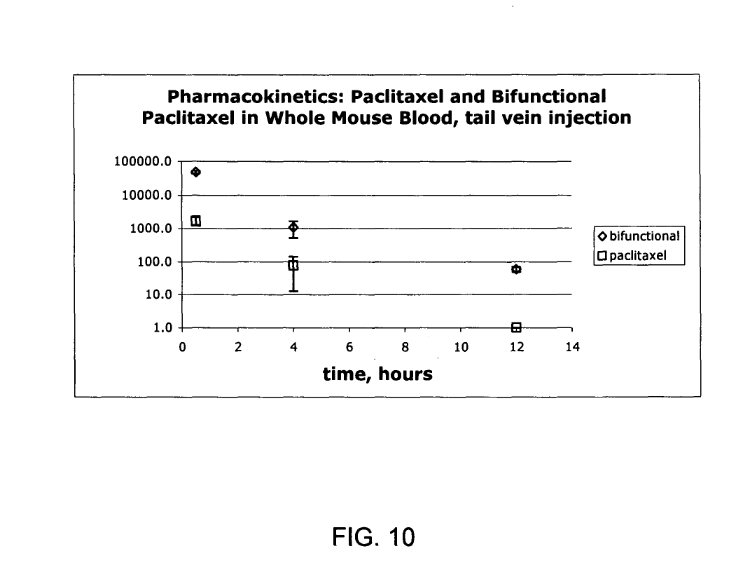

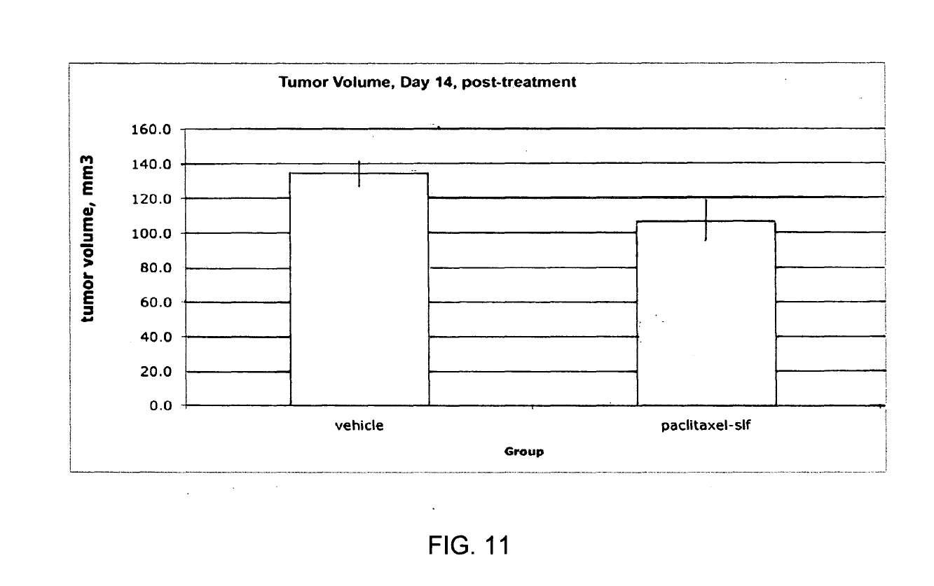

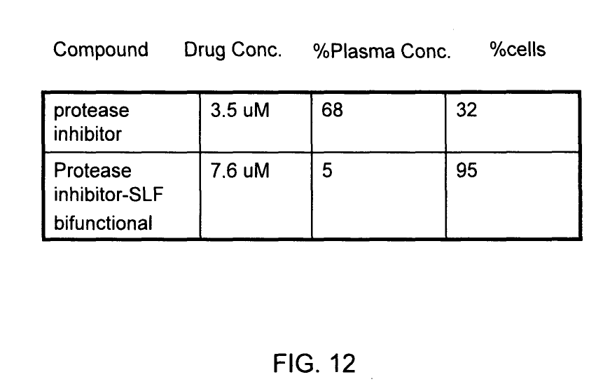

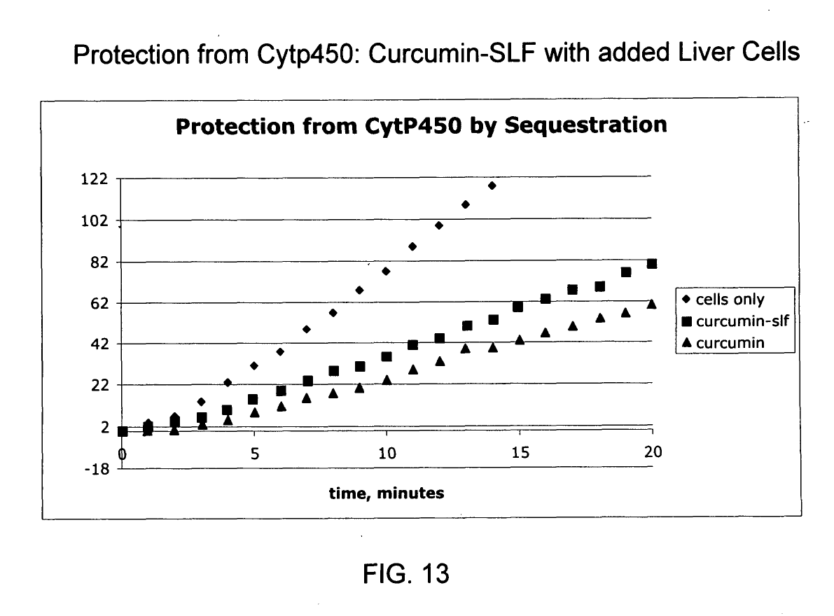

The recruiter moiety Z may be, for example, a derivative of FK506, which has high affinity for the FK506-binding protein (FKBP), as depicted for example in FIG. 1. There are many synthetic ligands for FKBP. The abundance of FKBP (as high as 100 μM) in blood compartments, such as red blood cells and lymphocytes, makes it likely that a significant proportion of a dose of bifunctional compounds comprising FK506 would partition initially into blood cells. The steric bulk conferred by FKBP would hinder an anti-infective therapeutic moiety from fitting into the binding pocket of intracellular enzymes (aldolases, hydroxylases, etc.) and so would prevent degradation via this class of enzymes. The high abundance (micromolar) and nanomolar dissociation constant would also help compete the drug moiety away from degradative enzymes. Moreover, the intracellular sequestration shields the drug moiety from extracellular degradation.

An inactive form of FK506 may be preferable in some applications to avoid the possibility of side effects due to the possible interaction of the active FK506-FKBP complex with calcineurin. It may be advantageous to use FKBP binding molecules such as synthetic ligands for FKBP (SLFs) described by Holt et al., supra. This class of molecule is lower molecular weight than FK506, and that is generally advantageous for drug delivery and pharmacokinetics. For illustrative purposes, some figures show examples of the use of FK506, though it should be understood that the same strategy can apply to other ligands of peptidyl prolyl isomerases such as the FKBP proteins and that ligands for other presenter proteins may be employed.

The value of FK506 and other FKBP binding moieties as further moieties Z in the invention is further supported by the following. FK506 (tacrolimus) is an FDA-approved immunosuppressant. It has been determined that FK506 can be readily modified such that it loses all immunomodulatory activity but retains high affinity for FKBP. FKBP is an abundant chaperone that is particularly prevalent (˜50 μM) in red blood cells (rbcs) and lymphocytes. The complex between FK506-FKBP gains affinity for calcineurin and inactivation of calcineurin blocks lymphocyte activation and causes immunosuppression.

This mechanism of action is derived from FK506's chemical structure. FK506 is by itself bifunctional; it has two non-overlapping protein-binding faces. One side binds FKBP, while the other binds calcineurin. This property provides FK506 with remarkable specificity and potency. Moreover, FK506 has a long half-life in non-transplant patients (21 hrs) and excellent pharmacological profile. In part, this is because FK506 is unavailable to metabolic enzymes via its high affinity for FKBP, which favors distribution into protected cellular compartments (72-98% in the blood). It can be expected that suitable bifunctional compounds with an FKBP-binding recruiter moiety will likewise possess some favorable characteristics of inactive FK506, namely, good pharmacokinetics and blood cell distribution.

The recruiter moiety Z may also derive, for example, from cyclosporins, rapamycin, geldanamycin, estrogen, progestin, testosterone, taxanes, colchicine, colcemid, nocadozole, cytochalasin, latrunculin, phalloidin, vinblastine, or vincristine.

Other exemplary targets for the recruiter moiety Z include cyclophilins A, B, C, D, or E, HCB (as disclosed in U.S. Pat. No. 5,196,352), HSP90, FKBP 12, FKBP 25,l FKBP 52, estrogen receptors, glucocorticoid receptors, androgen receptors, tubulin, and actin.

In situations where an extracellular rather than an intracellular bias may be desired, the recruiter moiety Z may derive, for example, from salicylate, dihydrotestosterone, bilirubin, hemin, myristilate, vitamin A, or vitamin D. Exemplary extracellular targets for the recruiter moiety Z include albumin, retinoic acid binding protein, vitamin A binding protein, vitamin D binding protein, or beta-2-macroglobulin.

As has already been noted, the presence of the recruiter moiety Z may have other favorable effects in addition to affecting the intracellular distribution of the bifunctional molecule relative to the drug from which the drug moiety derives. For example, deep joint infections are often difficult to treat but molecules such as SLF and macrolide antibiotics are known to penetrate well into biofilms and joints. The known anti-infectives are generally susceptible to metabolism and subsequent deactivation by hepatic first-pass or subsequent pass clearance mechanisms, which may also be alleviated by a suitable choice of recruiter moiety Z. The bifunctional drug may be particularly effective in reducing the duration of treatment required for the anti-infective agent since the bifunctional can evade mechanisms of drug resistance and target facultative parasites.

More generally, the recruiter moiety may affect favorably other pharmacokinetic properties of the drug moiety relative to the free drug from which it derives. The pharmacokinetic properties affected may include, for example, half-life, hepatic first-pass metabolism, volume of distribution, degree of blood protein binding, extent of P450 metabolism, and clearance. (For discussions of pharmacokinetic concepts one may refer, for example, to Brunton et al., supra, chapter 1, or to Leon Shargel & Andrew Yu, Applied Biopharmaceutics & Pharmacokinetics (4th ed. 1999).) Any of these may be different for the bifunctional molecule compared to the free drug from which the drug moiety X derives, even when the latter is administered by the same route and in a similar or identical formulation as the bifunctional molecule.

The linker L, if not simply a bond, may be any of a variety of moieties chosen so that they do not have an adverse effect on the desired operation of the two functionalities of the molecule and also chosen to have an appropriate length and flexibility. The linker may, for example, have the form F₁—(CH₂)ₙ—F₂ where F₁ and F₂ are suitable functionalities. A linker of this sort comprising an alkylene group of sufficient length may allow, for example, for the free rotation of the drug moiety even when the recruiter moiety is bound. Alternatively, a stiffer linker with less free rotation may be desired. The hydrophobicity of the linker is also a relevant consideration. FIG. 6A depicts some precursors which may be used for the linker (with the carboxyl functionality protected).

It is desirable for at least some embodiments of the present invention that the binding of the recruiter moiety Z to a common protein be such as to sterically hinder the activity of common metabolic enzymes such as CYP450 enzymes when the bifunctional compound is so bound. Persons of skill in the art will recognize that the effectiveness of this steric hindrance depends, among other factors, on the conformation of the common protein in the vicinity of the recruiter moiety's binding site on the protein, as well as on the size and flexibility of the linker. The choice of a suitable linker and recruiter moiety may be made empirically or it may be made by means of molecular modeling of some sort if an adequate model of the interaction of candidate recruiter moieties with the corresponding common proteins exists.

A further aspect of the invention is that the linker has been chosen to enhance solubility of the bifunctional compound relative to either the recruiter moiety or drug moiety. The linker would typically be polar, but avoids moieties which hinder permeability across membranes. Some examples of membrane permeable, polar moieties are molecules with side chains containing lysine, arginine, guanidine, ethylamine, and other basic moieties. The enhanced solubility is helpful in avoiding toxic solvents such as cremaphor. Additionally, kinase inhibitors often have poor solubility due to their hydrophobicity. Since this hydrophobicity is helpful for target binding, enhancing solubility with a polar linker and the addition of a recruiter moiety to assist targeting will enhance kinase efficacy and create a bifunctional entity which is soluble in aqueous solutions.

Examples of linkers which are believed to enhance solubility are given in FIG. 6B. In that figure, R¹, R², R³ may be arginine, guanidine, lysine or ethylamine. More generally, the R¹, R², R³ substituents may be basic moieties with a pKₐ of at least 7.5.

In this aspect of the invention, the drug moiety X need not be an anti-infective. It may belong to a wide variety of therapeutic categories including, but not limited to: analeptic agents; analgesic agents; anesthetic agents; anti-arthritic agents; respiratory drugs, including anti-asthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; anti-inflammatory agents; antimigraine preparations; antinauseants; antiparkinsonism drugs; antipruritics; antipsychotics; antipyretics; antispasmodics; antitubercular agents; anti-ulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; sympathomimetics; tranquilizers; and vasodilators including general coronary, peripheral and cerebral.

In a further embodiment of the invention, a free derivative or analog of the drug moiety, for example the drug from which the drug moiety is derived, is administered in free form jointly with the administration of a bifunctional. This may be useful, for example, where the targeted parasite is a facultative intracellular parasite, and there is a need for targeting to the parasite in its extracellular as well as intracellular state.

In this aspect of the invention, the drug moiety X need not be an anti-infective, and may belong, for example, to any of the therapeutic categories listed above.

In a further embodiment of the invention, a free analog or derivative of the recruiter moiety may be given with the bifunctional. This may be done, for example, to enhance distribution into a biofilm or enhance susceptibility of a drug target to the combination of bifunctional with free recruiter. Co-administration of free recruiter may be advantageous since the free recruiter may, for example, block binding of the bifunctional to the a drug efflux pump or enhance the susceptibility of a drug target. Wang, L. Clin. and Experiment. Pharmacol. & Phys. 27, pp. 587-593 (2000). For example, FK506 has also been found to act synergistically with the anti-infective fluconazole. C. Onyewu et al., “Ergosterol biosynthesis inhibitors become fungicidal when combined with calcineurin inhibitors against Candida albicans, Candida glabrata, and Candida krusei,” Antimicrob. Agents Chemother. 47, 956-964 (2003). FK506 has also been found to reverse multidrug resistance in tumor cells. M. Naito et al., “Reversal of multidrug resistance by an immunosuppressive agent FK-506,” Cancer Chemother. Pharmacol. 29, pp. 195-200 (1992).

In this aspect of the invention, the drug moiety X need not be an anti-infective, and may belong, for example, to any of the therapeutic categories listed above.

In a further aspect of the invention, a bifunctional compound comprising a anti-infective therapeutic moiety is formulated, for example in the form of a tablet, capsule, parenteral formulation, to make a pharmaceutical preparation. The pharmaceutical preparation may be employed in a method of treating a patient having cancer against which the anti-infective therapeutic moiety is effective. For example, if the anti-infective therapeutic moiety is effective against Aspergillus fumigatus, the pharmaceutical preparation may be administered to a patient suffering from invasive aspergillosis. The bifunctional compound may be delivered in a carrier such as a liposome, nanoliposome, or other common carriers such as pegylated liposomes.

For the preparation of a pharmaceutical formulation containing bifunctional compounds as described in this application, a number of well known techniques may be employed as described for example in Remington: The Science and Practice of Pharmacy, Nineteenth Ed. (Easton, Pa.: Mack Publishing Company, 1995) and Modern Pharmaceutics (Gilbert S. Banker & Christopher T. Rhodes eds., 3d ed. 1996).

In a further aspect of the invention, a bifunctional compound is formulated as part of a controlled release formulation. The bifunctional compound is as above, comprising a drug moiety, a linker, and a recruiter moiety. In this aspect of the invention, a drug moiety may be an anti-infective therapeutic or a different type of drug.

A compound of the disclosure may be administered in the form of a salt, ester, amide, prodrug, active metabolite, analog, or the like, provided that the salt, ester, amide, prodrug, active metabolite or analog is pharmaceutically acceptable and pharmacologically active in the present context. Salts, esters, amides, prodrugs, active metabolites, analogs, and other derivatives of the active agents may be prepared using standard procedures known to those skilled in the art of synthetic organic chemistry and described, for example, by J. March, Advanced Organic Chemistry: Reactions, Mechanisms and Structure, 5th Ed. (New York: Wiley-Interscience, 2001). Furthermore, where appropriate, functional groups on the compounds of the disclosure may be protected from undesired reactions during preparation or administration using protecting group chemistry. Suitable protecting groups are described, for example, in Green, Protective Groups in Organic Synthesis, 3rd Ed. (New York: Wiley-Interscience, 1999).

It is to be understood that while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

The following examples are put forth so as to provide those of ordinary skill in the art with a complete disclosure and description of how to implement the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric.

### Example 1

### Models of Prosthetic Joint Infection

Bifunctional therapy for a prosthetic joint infection was studied in a rabbit model as follows: partial knee arthroplasty was performed with a silicon elastomer implant. After closing the wound, an inoculum of 5×10⁶ cfu of methicillin resistant S. aureus is injected into the replacement joint. Infection develops reliably in this model. Infection normally progresses into the tibia and produces chronic osteomyelitis, verified by magnetic resonance imaging (MRI), pathology, and histopathologic variation. ¹⁴C-sparfloxacin and ¹⁴C-bifunctional sparfloxacin are administered at doses up to 50 mg/kg and autoradiography is used to assess localization of administered drug to the site of infection (A. Cremieux et al., Clin. Infect. Dis., 25, pp. 1295-1302 (1997)). The seven-day treatment is started 15 days after the S. aureus inoculation. Three weeks after the end of treatment, animals are sacrificed, the bone is pulverized, and colony forming units (CFU) per gram of bone are analyzed. The CFU of the parent compound and bifunctional are compared to assess efficacy.

### Example 2

### Bifunctional Efficacy in an In Vitro Facultative Parasite Model

To screen bifunctional libraries of voriconazole compounds against the facultative intracellular parasites Candida albicans and Candida krusei, radiolabeled voriconazole and voriconazole bifunctional compound are made at 50 μCi/mg. Human polymorphonuclear leucocytes (HPMN's) are incubated in buffer with concentrations of voriconazole ranging from 1-40 mg/L. After incubation at 37° C., cells are pelleted through a silicon oil barrier and scintillation counting is used to determine the accumulation ratio, expressed as cellular to extracellular (or C/E). Incubation times for the uptake are 1, 5, 10, 20, and 60 minutes.

To study the efflux of HPMN's containing parent and bifunctional drugs, suspensions of HPMN's containing radiolabeled drug and radiolabeled bifunctional drug are made and the C/E ratio is determined by scintillation counting. To evaluate intracellular efficacy, yeast suspension and HPMN's are incubated, typically for one hour. Extracellular blastopores are removed by centrifugation, and cells are resuspended in RPMI medium. Next, different concentrations of parent and bifunctional compound are added (1, 2, and 5 mg/mL), and cells are incubated at 37° C. for three hours. Cells are then lysed in distilled water, and samples are diluted and plated on Sabourand agar. Colonies are counted after 2 days of incubation at 37° C. The data is expressed as percentage of Candida surviving compared with the bifunctional and parent compound.

### Example 3

### Synthesis of Ertapenem-SLF Bifunctional

The linkers shown in FIG. 6 may be coupled to FK506 or SLF via EDC-mediated amide formation followed by deprotection of the newly installed amine. This acid is then used for conjugation to the ertapenem molecule as shown in FIG. 7. The linker can be readily altered to enhance solubility or other physical characteristics of the bifunctional compound. The linker may contain a tertiary amine in some instances to help inhibit cell wall formation in a parasite. The linker must cross cell membranes in the context of the bifunctional molecule. In one preferred embodiment, the linker must permit simultaneous binding of the recruiter moiety and drug moiety by the bifunctional.

### Examples 4

### Synthesis of Antiinfective-SLF Conjugate Mini-Library

The syntheses of anti-infective-SLF conjugates may proceed in a fashion generally similar to that employed for the FK506-based molecule, as shown in FIG. 7. Linkers as shown in FIG. 6 may be employed to generate a small bifunctional library. Linker choice can be important since it can effect compound solubility, transport from the small intestine into the circulation, equilibrium between target and non-target protein binding, efflux via the p-glycoprotein pump, and intra- vs. extracellular distribution.

### Example 5

### Test of Efficacy of Bifunctional Compounds in an Animal Model of Bacterial Pneumonia

For preclinical testing of bifunctional anti-infective drugs, female Hartley strain guinea pigs (weight 350-400 grams) are used. Animals are housed in regulation cages and given food and water ad libitum. A bifunctional library of amoxicillin, cefotaxime, and meropenem is screened for efficacy against penicillin insensitive Streptococcus pneumoniae in non-neutropenic animals. Controls are parent compound or animals receiving vehicle only. MIC's are determined in advance of the in vivo study using a microdilution method in Mueller-Hinton broth to which 5% lysed sheep blood is added. Each microplate well contains a two-fold serial dilution of antibiotic and a final bacterial concentration of 5×10⁴ CFU per well. Plates are incubated at 37° C. for 18 hours, and the MIC is defined as the lowest concentration of antibiotic which gave no detectable growth.

For the pneumonia model, the animals are inoculated in the neck after anesthetization by subcutaneous injection, and each pig's trachea is exposed by a vertical midline incision. The inoculum is injected intratracheally with a 25-gauge needle, and the incision is closed with steel wound clips. To prepare inoculum, an overnight culture of S. pneumoniae is grown in Todd-Hewitt broth and frozen in 1 mL aliquots at −70° C. For the experiments, aliquots are thawed, and 2 mL of suspension is used to seed 50 mL of fresh broth, followed by overnight incubation at 35° C. in a 5% CO₂ atmosphere. Cultures are then centrifuged and resuspended in fresh broth to obtain concentrations that are 1, 5, 10, and 25-fold higher than the uncentrifuged culture. Animals still living are sacrificed at 46 hours by sodium pentobarbitol injection, lungs are aseptically removed and washed in phosphate-buffered saline, and the lungs are homogenized with glass tissue grinders. Homogenates are serially diluted into broth, and 100 μL were plated in duplicate onto 5% blood agar, and plates are incubated at 35° C. for 24 hours in 5% CO₂ to determine the CFU of S. pneumoniae present.

Once the infection model is established, animals are treated as follows: two doses of each test antibiotic is administered to different test groups as well as a vehicle control with 5 or 6 animals per group. Typical dosages are 50 and 200 mg/kg and are administered at 1, 9, 17 and 25 hours after inoculation. After 46 hours, surviving animals are sacrificed and lung CFU's are determined as described above.

### Example 6

### Test of Efficacy of Bifunctional Compounds in an Animal Model of Invasive Aspergillosis in an Immunocompromised Guinea Pig Model

A library of voriconazole bifunctionals is tested in an invasive aspergillosis model using isolate P171 of Apsergillus fumigatus. The isolate is grown on Sabourand dextrose slants at 37° C. for 24 hours and prepared for injection at 10⁶ condidia/mL by hemacytometer count. Male Hartley Guinea pigs were immunocompromised using cyclophosphamide (W. Kirkpatrick, Antimicrob. Agents Chemother. 50, 2006, pp 1567-1569). The animals are injected through the saphenous vein with 10⁶ A. fumigatus conidia. Systemic infection including the lung, kidney, liver, and brain develop quickly. Antifungal therapy is initiated 24 hours after inoculation and continues for 5 days. The parent voriconazole and bifunctional voriconazole derivatives are administered by tail vein injection at 5 mg/kg twice per day with 6 animals per group. Postmortem or 96 hours after the completion of treatment, organ cultures are performed and survival and colony counts/gram tissue are tabulated.

### Example 7

### Test of Efficacy of Bifunctionals Against Intraphagocytic Staphylococcus Aureus in an In Vitro Model and % Serum-Bound Bifunctional

For intracellular infection, THP-1 myelomonocytic cells are used. For S. aureus infection, opsonization was performed with non-decomplemented, freshly thawed human serum diluted 1:10 in serum free culture medium, RPMI 1640. Phagocytosis takes place at a bacteria:macrophage cell ratio of 4:1. Non-phagocytosed bacteria are eliminated via centrifugation at room temperature and remaining cells contain intracellular, phagocytosed bacteria. Intracellular concentrations of antibiotic are determined in an activity assay by assessing the MIC obtained from lysed THP-1 cell extracts against a reference Escherichia coli strain by comparing prior MIC values obtained in cell free media with the MIC of sonicated cell suspensions. Bifunctional carbapenem MIC and minimum bactericidal concentrations (MBC) are compared with MIC and MBC for the parent compounds. THP-1 cell protein concentrations were measured using the Folin-Ciocalteu method to determine the cellular concentrations of antibiotic per cell. Bifunctional compounds with the lowest MIC and MBC will be tested further in animal models to assess efficacy.

In addition to MIC and MBC values, amounts of protein bound drug were determined by dialysis to determine whether the bifunctional compound had more or less affinity for serum albumin than the parent compound. A dialysis cutoff filter of 6000-8000 molecular weight is used and the antibiotic is placed in a serum free cell. The membrane is soaked in three water baths and three baths of phosphate buffered saline (PBS). Equilibrium required about 32 hours at 37° C. at a rotation of 8 rpm. The final concentration of antibiotic in the dialysis membrane is determined by HPLC. To calculate the percentage of serum-bound drug outside the dialysis chamber, the following formula is used: 100−[(concentration in serum free cell×2)/initial concentration)].

### Example 8

### Protection of Bifunctional from Extracellular Enzymatic Degradation in a Whole Blood Sample

To determine the extent of the ability of intracellular sequestration to protect bifunctional compound from degradation, bifunctional compounds are incubated in a sample of pure plasma and, in parallel, in a sample of whole blood. Aliquots of plasma and whole blood are sampled at different time points to determine the amount of compound remaining over time. Liquid chromatography-mass spectroscopy (LC-MS) is used to verify the identity of metabolites to distinguish different potential breakdown products from esterases, P450 enzymes, among others. Appropriate extraction controls are included with known amounts of compound to account for yield efficiency and compound loss prior to the analysis. For the whole blood sample, the blood is further separated into plasma and whole blood to assess the % of intracellular sequestration. Data for a sample bifunctional is given in FIG. 9.

Similarly, a commercial Invitrogen P2856 assay was used to test for degradation via CYP3a4 in accordance with the manufacturer's directions in the presence of red blood cells. In the presence of 1 μM FKBP, a bifunctional curcumin moiety is completely protected from degradation via the CYP3a4 as shown in FIG. 5. The monofunctional curcumin compound is >70% degraded under the same condition. In the absence of FKBP, the bifunctional is over 70% degraded by the CYP450 enzyme. This cell-free demonstration suggests that if drugs are sequestered inside cells, they will similarly be protected from extra-cellular CYP450. Data illustrating this is given in FIG. 10. The area under the curve for the paclitaxel bifunctional is over 25 times the area under the curve for the monofunctional in data obtained from tail vein injections of mice when measured in whole blood. The area under the curve for the bifunctional is over 60 times the area under the curve for the monofunctional in data obtained from tail vein injections of mice when measured in plasma (data not shown on this curve).

Additionally, FIG. 11 illustrates the in vivo efficacy of a paclitaxel bifunctional against a human xenograft tumor cell line MDA-MB-435 in mice. The data show that the drug activity is maintained in the presence of the bifunctional modification.

### Example 9

### Verifying Intracellular Distribution

The recruiter moiety choice is commonly used to bias extra and intracellular distribution. The bias is dependent on the choice of drug target. Drugs such as insulin operate on extracellular receptors and there is no efficacy advantage to internalizing the protein to the intracellular space, although there is still an advantage of creating a sustained release moiety and protecting the bifunctional from degradation. However, many anti-infective therapeutics such as ciprofloxacin bind to an intracellular component such as DNA topoisomerase, thus making an intracellular bias desirable. Nonetheless, overly biasing the distribution in the intracellular case will make it impossible for the drug to spread its effect over a large number of cells given the limited dose amount (typically 130 mg/m² for topotecan in humans, for example). Overbiasing the drug in the extracellular case will make it difficult to target the drug to specific locations if the therapeutic must cross cell membranes to achieve effective transport.

So, the tuning of the extracellular and intracellular distribution is important, and must be engineered using both the K_{D} and k_{off} parameters of the bifunctional binding to the drug target and bifunctional binding to the recruited biomoiety. To target blood cells intracellularly, it is desirable to target specific intracellular proteins such as FKBP-12, Erp-1, or Es-1. Recruiter moieties are desirably optimized to cross the cell membrane. Analysis of the selected pool of recruiter moieties may proceed by the analysis below.

Once a decision is made for either an extra- or intra-cellular bias, the recruiter moiety is chosen accordingly, Moreover the recruiter moiety is designed to strike the correct balance to allow cell membrane transport where necessary. The bias may determined kinetically as described above by determining affinity and kinetic parameters for the bifunctional with respect to the drug target and recruited biomoiety. Kinetic and endpoint distributions in plasma and whole blood are determined by liquid chromatography and mass spectroscopy.

A typical protocol for the determination of compartmentalization into whole blood is as follows:

1. Add drug (10 mM in DMSO) to 250 μl whole blood for a final concentration of 10.0 & 30.0 μM
2. Incubate in shaker at 37° C. for 240 minutes
3. Separate plasma by centrifuging at 1,000×g (3706 rpm) for 15 minutes at 4.0° C. (Plasma samples may be stored at −80° C. at this point.)
4. Transfer 75 μl of plasma to new microfuge tubes
5. Extract drug by adding 1 ml ethyl acetate 2× and vortexing vigorously
6. Centrifuge at 12,000×g (13,200 rpm) for 7 minutes at 4.0° C.
7. Transfer supernatants to fresh glass vials
8. Evaporate to dryness using RotoVap
9. Reconstitute residue in 500 μl of acetonitrile/0.1% acetic acid
10. Inject onto LCMS

Plasma samples are run in the same manner to determine the ratio of compound in whole blood vs. plasma where the plasma sample represents the extra-cellular blood fraction and whole blood samples contain both the intra- and extra-cellularly distributed drug.

We claim:

1. A method of enhancing the efficacy of an anti-infective therapeutic agent against an obligate or facultative intracellular parasite in a host, the method comprising:
(a) administering to the host an effective amount of a bifunctional compound of less than about 5000 Daltons comprising the therapeutic or an active derivative, fragment or analog thereof and a protein binding moiety,
(b) wherein the protein binding moiety binds to at least one intracellular protein,
(c) wherein the bifunctional compound has at least one pharmacokinetic property on administration to the host which is different from the same pharmacokinetic property of the therapeutic itself, and
(d) wherein the bifunctional compound has a higher intracellular concentration as compared to the therapeutic itself in order to enhance the efficacy of the anti-infective therapeutic against the obligate or facultative intracellular parasite.

2. The method according to claim 1, wherein the pharmacokinetic property is selected from the group consisting of half-life, hepatic first-pass metabolism, volume of distribution, degree of blood protein binding, extent of P450 metabolism, and clearance.

3. The method according to claim 1, wherein the bifunctional compound is administered as a pharmaceutical preparation.

4. The method according to claim 1, wherein the host is a mammal or bird.

5. The method according to claim 1, wherein the protein binding moiety has a mass of less than 1200 Daltons and binds to a peptidyl prolyl isomerase.

6. The method according to claim 1, wherein the therapeutic agent is a carbepenem.

7. The method according to claim 1, wherein the therapeutic agent is meropenem.

8. The method according to claim 1, wherein the therapeutic agent is a triazole.

9. The method according to claim 1, wherein the therapeutic agent is voriconazole.

10. The method according to claim 1, wherein the therapeutic agent is amphotericin B.

11. The method according to claim 1, wherein the therapeutic agent is caspofungin.

12. The method according to claim 1, wherein the therapeutic agent is a cephalosporinase.

13. The method according to claim 1, wherein the therapeutic agent is antifungal.

14. The method according to claim 1, where the therapeutic agent is antibacterial.

15. The method according to claim 1, wherein the therapeutic agent is doripenem.

16. A method for improving the solubility of a therapeutic, the method comprising:
(a) providing an effective amount of a bifunctional compound of less than about 5000 Daltons comprising (a1) a drug moiety which includes the therapeutic or an active derivative, fragment or analog thereof, (a2) a protein binding moiety, and (a3) a linker joining the drug moiety to the protein binding moiety,
(b) wherein the moiety binds to at least one intracellular protein,
(c) wherein the bifunctional compound has at least one pharmacokinetic property on administration to the host which is different from the same pharmacokinetic property of the therapeutic itself, and
(d) wherein the linker causes the bifunctional compound to have a greater aqueous solubility than the therapeutic or greater aqueous solubility than a second bifunctional compound comprising the drug moiety and the protein binding moiety but no linker.

17. The method of claim 16 where the linker comprises a polymeric chain with a pKₐ between about 7 and about 14.

18. The method of claim 16 where the linker comprises a polymer containing at least one of the following monomers: lysine, histidine, arginine, guanidine, or ethylamine.

19. The method of claim 16 where the linker comprises a plurality of D-amino acids with a side chain consisting of at least one lysine, histidine, arginine, or guanidine monomer.

20. The method of claim 16 where the drug moiety is a kinase inhibitor.

21. The method of claim 16, wherein the bifunctional compound comprises a tertiary amine function.

22. The method of claim 16, wherein the drug moiety is a carbapenem.

23. A method for treating in a patient in need of treatment a medical condition which is treatable by a therapeutic, the method comprising:
(a) providing an effective amount of a bifunctional compound of less than about 5000 Daltons comprising (a1) a drug moiety which includes the therapeutic or an active derivative, fragment or analog thereof, (a2) a protein binding moiety, and (a3) a linker joining the drug moiety to the protein binding moiety,
(b) wherein the moiety binds to at least one intracellular protein,
(c) wherein the bifunctional compound has at least one pharmacokinetic property on administration to the host which is different from the same pharmacokinetic property of the therapeutic itself, and
(d) administering both the bifunctional compound and the therapeutic to the patient.

24. A method for treating a condition which is treatable by a therapeutic, the method comprising:
(a) providing an effective amount of a bifunctional compound of less than about 5000 Daltons comprising (a1) a drug moiety which includes the therapeutic or an active derivative, fragment or analog thereof, (a2) a protein binding moiety, and (a3) a linker joining the drug moiety to the protein binding moiety,
(b) wherein the moiety binds to at least one intracellular protein,
(c) wherein the bifunctional compound has at least one pharmacokinetic property on administration to the host which is different from the same pharmacokinetic property of the therapeutic itself, and
(d) administering to the patient both the therapeutic and a derivative or analog of the protein-binding moiety.
